(19) United States
(12) Plant Patent Application Publication
(10) Pub. No.: US 2002/0059668 P1
(43) Pub. Date: May 16, 2002

(54) VERBENA PLANT NAMED 'SUNMAREF TP-SAP'

(76) Inventor: SHIGA YOMO, YASU-GUN (JP)

Correspondence Address: BENTON S DUFFETT JR, BURNS DOANE SWECKER & MATHIS L L P, P O BOX 1404, ALEXANDRIA, VA, 223131404

(21) Appl. No.: 09/328,271

(22) Filed: Jun. 17, 1999

(51) Int. Cl. A01H 5/00

(52) U.S. Cl. PLT/308

(57) ABSTRACT

Disclosed herein is a verbena plant named ‘SUNMAREF TP-SAP’s has spreading growth habit having long stems and spreading area of plant of broad. The plant has abundant branching and plentiful number of flower in a spike, and great profusion of blooms. The Blooming period is April to November and flowering duration is long, and the whole bush remains in bloom for a considerable period of time. The flower size is medium and the petal color of flower is strong purplish pink color. The plant is highly tolerant to cold, drought, rain and heat, and has high resistance to pests and diseases, particularly powdery mildew.

### BACKGROUND OF THE VARIETY

The present invention relates to a new and distinct variety of verbena plant obtained from a crossing a wild type of verbena plant Verbena calliantha f. rosea (♀) native to Brazil and ‘Rainbow carpet red’ (♂).

The verbena is a very popular plant and is used for flower bedding and potting in the summer season. There are only a few varieties of the verbena plant which have a spreading growth habit, much branching, a large number of flowers in a spike and which have a high resistance to rain, heat, cold, and diseases. Accordingly, this invention was aimed at obtaining a new variety having a spreading growth habit, strongly branching, many number of flowers in a spike, high tolerance to heat, rain, drought and cold, and resistance to diseases and pests, and also having a strong purplish pink color petal.

The new variety of verbena plant according to this invention originated from crossing of a wild type of verbena plant Verbena calliantha f. rosea (♀) native to Brazil and ‘Rainbow carpet red’ (♂).

First of all, 35 seedlings were obtained from crossing a wild type of verbena plant, Verbena calliantha f. rosea, as female parent and ‘Rainbow carpet red’ as pollen parent in March of 1996. From this crossing one seedling was selected in view of spreading growth habit and flower color. This one selected seedling was propagated by cutting and grown and was carried out a trial by flower potting and bedding, and then the botanically characteristics of the seedling was examined, using similar variety ‘Rainbow carpet red’ for comparison from the spring of 1997 to the autumn of 1998. As a result, it was concluded that this verbena is distinguishable from any other variety, whose existence is known to us, uniform and stable in its characteristics, and this new variety of verbena plant was named ‘SUNMAREF TP-SAP’.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plant (J.H.S.. Color Chart) is also added for reference.

The female parent used in the obtaining of this new variety ‘SUNMAREF TP-SAP’ was a wild type of verbena native (Verbena calliantha) to South Brazil which introduced to Japan in 1995. This wild type of verbena plant is presently maintained at the Plant Biotechnology Laboratory of SUNTORY Ltd., residing at 2913-1 Torihara, Hakusyu-cho, Kitakoma-gun, Yamanashi-ken, Japan. The main botanical characteristics of this pollen parent are as follows.

Plant:

Growth habit.—Slightly erected spreading.

Plant extension.—100-150 cm.

Plant height.—5-8 cm.

Stem:

Diameter.—1.0-2.0 mm.

Anthocyanin pigmentation.—Present.

Branching.—Medium.

Subterranean stem.—Absent.

Pubescence.—Present.

Length of internode.—2.0-5.0 cm.

Leaf:

Phyllotaxis.—Opposite.

Shape of blade.—Narrow lanceolate.

Length.—2.0-4.0 cm.

Width.—1.0-2.0 cm.

Depth of incision.—Deep.

Color.—Dark green(R.H.S. 137A, J.H.S. 3707).

Pubescence.—Sparse.

Flower:

Facing direction.—Upward.

Outward curvature of petal.—Slightly curved.

Diameter.—1.5-2.0 cm.

Height.—15-20 mm.

Color.—Deep purplish pink (R.H.S. 66C, J.H.S. 9505).

Color intensity.—Absent.

Overlapping of petals.—Separate.

Spike.—20-25 mm in length; and 30-40 mm in diameter.

Calyx.—1.0 cm in length.

Anthocyanin pigmentation of calyx limb.—Present.

Peduncle.—1-2 mm in thickness; and 4.0-5.0 cm in length.

Number of flowers.—Plentiful (13-15).

Reproductive organs.—1 pistil and 5 stamens.

Flower fragrance.—Absent.

Flowering duration.—Short.

Physiological and ecological characteristics: High Resistance to diseases and pests, high tolerance to heat and moderate tolerance to cold.

The ‘Rainbow carpet red’ used as pollen parent in the obtaining of this new variety ‘SUNMAREF TP-SAP’, was bred by Takii Seed Corp., Japan and is on the market. The ‘Rainbow carpet’ series includes ‘Rainbow carpet bright purple’, ‘Rainbow carpet white’, and the like. The main botanical characteristics of ‘Rainbow carpet red’ are as follows.

Plant:

Growth habit.—Semi-erect.

Plant extension.—25-30 cm.

Plant height.—20-25 cm.

Stem:

Diameter.—1.7-1.9 mm.

Anthocyanin pigmentation.—Absent.

Branching.—Medium.

Subterranean stem.—Absent.

Pubescence.—Sparse.

Length of internode.—3.0-3.5 cm.

Leaf:

Phyllotaxis.—Opposite.

Shape of blade.—Cordate.

Length.—2.0-2.5 cm.

Width.—1.5-2.0 cm.

Depth of incision.—Deep.

Color.—Dark green (R.H.S. 137B, J.H.S. 3716).

Pubescence.—Sparse.

Flower:

Facing direction.—Upward.

Outward curvature of petal.—slightly curved.

Diameter.—1.2-1.7 cm.

Height.—13-15 mm.

Color.—Vivid red (R.H.S. 52A, J.H.S. 0106).

Color intensity.—Absent.

Overlapping of petals.—Opened.

Spike.—25-30 mm in length and 30-35 mm in diameter.

Calyx.—0.5-1.0 cm in length.

Anthocyanin pigmentation of calyx limb.—Present.

Peduncle.—1.7-1.8 mm in thickness and 4.0-6.0 cm in length.

Number of flowers.—Few (7-11).

Reproductive organs.—1 pistil and 5 stamens.

Flower fragrance.—Absent.

Flowering duration.—Medium.

Physiological and ecological characteristics: Moderate tolerance to heat, cold, and pests and low resistance to diseases.

This new variety of verbena plant named ‘SUNMAREF TP-SAP’ was asexually reproduced by cutting at the aforementioned the Hakusyu Research Center of SUNTORY Ltd., residing at 2913-1 Torihara, Hakusyu-cho, Kitakomagun, Yamanashi-ken, Japan, and the homogeneity and stability thereof were confirmed.

### SUMMARY OF THE VARIETY

This new variety of verbena plant has spreading growth habit having long stems and spreading area of plant of broad. The plant has abundant branching and a large number of flowers in a spike, and great profusion of blooms. The Blooming period is April to November and flowering duration is long, and the whole bush remains in bloom for a considerable period of time. The flower size is medium and the petal color of flower is strong purplish pink. The plant is highly tolerant to cold, heat, rain and drought and high resistance to pests and diseases, particularly powdery mildew.

### BRIEF DESCRIPTION OF THE DRAWINGS

FIG. 1 is photograph giving a partial view of the new variety of verbena plant named ‘SUNMAREF TP-SAP’.

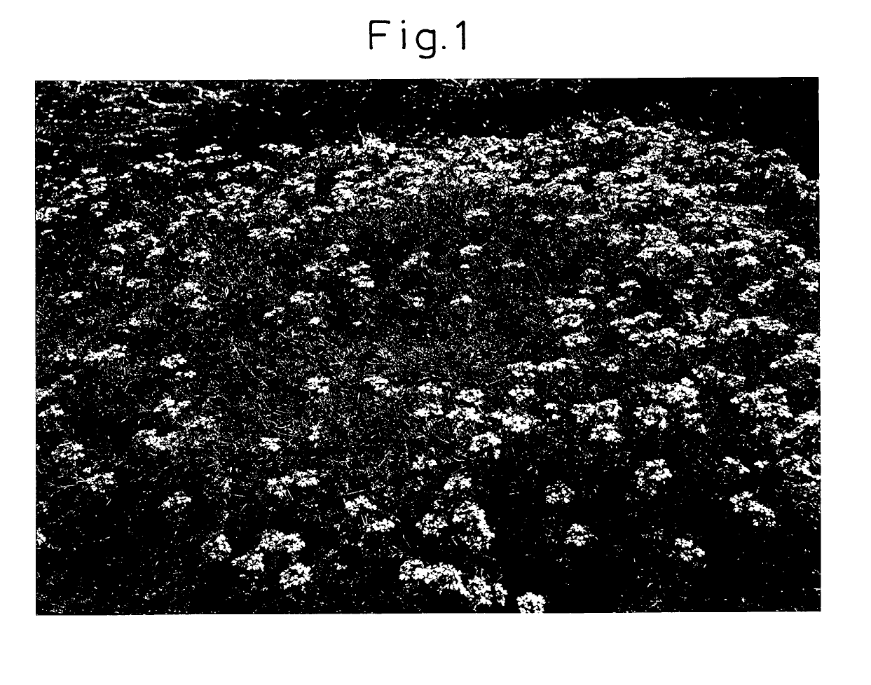

FIG.2 is a photograph of flowers of the new variety of verbena plant named ‘SUNMAREF TP-SAP’.

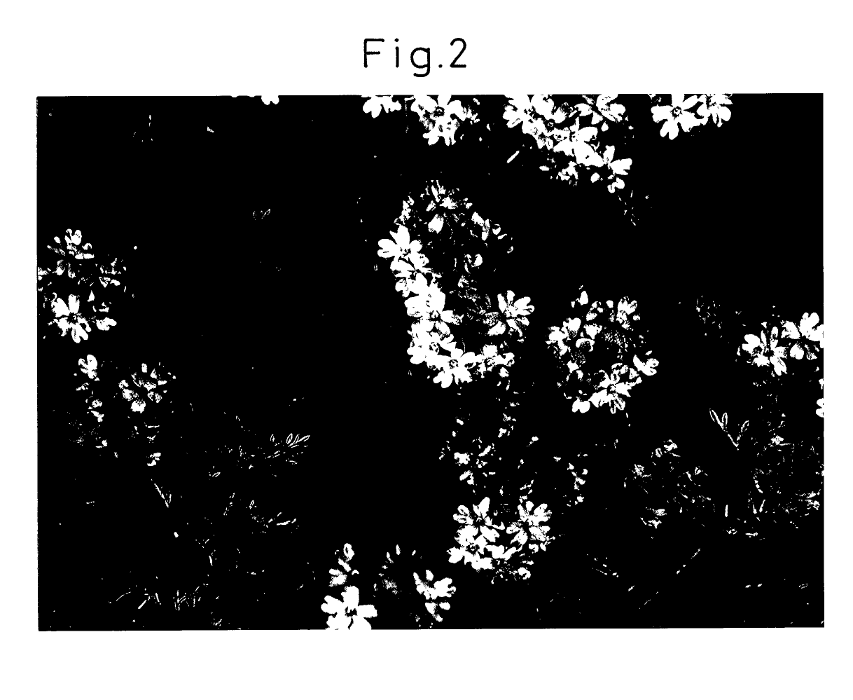

### DESCRIPTION OF THE VARIETY

The botanical characteristics of the new and distinct variety of verbena plant named ‘SUNMAREF TP-SAP’ is as follows.

Plant:

Growth habit.—Spreading.

Plant extension.—80-100 cm.

Plant height.—10-20 cm.

Growth.—Very vigorous with abundant branching and great profusion of blooms; the whole bush remaining in bloom for considerable period of time.

Stem:

Diameter.—2.0-3.0 mm.

Anthocyanin pigmentation.—Present.

Branching.—Abundant.

Subterranean stem.—Absent. But when the stems contact the surface of soil, the nodes take root in the ground and the plant has its root fastened.

Pubescence.—Present.

Length of internode.—2.0-3.0 cm.

Leaf:

Phyllotaxis.—Opposite.

Shape of blade.—Cordate.

Length.—3.2-3.6 cm.

Width.—1.5-2.2 cm.

Depth of incision.—Deep.

Color.—Dark green (R.H.S. 132A, J.H.S. 4307).

Pubescence.—Present.

Flower:

Facing direction.—Upward.

Outward curvature of petal.—Slightly curved.

Diameter.—1.3-1.5 cm.

Height.—12-14 mm.

Color.—Strong purplish pink (R.H.S. 55A, J.H.S. 9705).

Color intensity.—Absent.

Overlapping of petals.—Opened.

Spike.—30-50 mm in length; and 30-40 mm in diameter.

Calyx.—0.7-0.8 cm in length.

Anthocyanin pigmentation of calyx limb.—Present.

Peduncle.—1 mm in thickness; and 4.0-5.0 cm in length.

Number of flowers.—Medium (7-12).

Reproductive organs.—1 pistil and 5 stamens.

Flower fragrance.—Absent.

Flowering duration.—Long.

Physiological and ecological characteristics: High resistance to diseases and pests, particularly powdery mildew. High tolerances to heat, cold, rain and drought.

This new variety of verbena plant is most suitable for flower bedding and potting, particularly in planters, and is further excellent for ground cover.

The plant of this new variety, ‘SUNMAREF TP-SAP’ is presently planted and maintained at the Hakusyu Research Center of SUNTORY Ltd., residing at 2913-1 Torihara, Hakusyu-cho, Kitakoma-gun, Yamanashi-Ken, Japan.

1. A new and distinct variety of verbena plant named ‘SUNMAREF TP-SAP’ , substantially as herein illustrated and described, characterized particularly as to novelty by (A) being a spreading growth habit having long stems, (B) an abundant branching and a large number of flowers in a spike and a great profusion of blooms, long flowering duration, and the whole bush remaining in bloom for a considerable period of time, (C) flower size is medium and the petal color of flower is strong purplish pink color, (D) a high torelance to rain, heat, drought, cold, and a high resistance to diseases and pests, particularly powdery mildew.
